(19) United States
(12) Patent Application Publication
(10) Pub. No.: US 2011/0290740 A1
(43) Pub. Date: Dec. 1, 2011

(54) WASTE TREATMENT AND DISINFECTION UNIT

(75) Inventors: Joseph B. Dooley, Kingston, TN (US); Jeffrey G. Hubrig, Knoxville, TN (US)

(73) Assignee: INNOVATION SERVICES, INC., Knoxville, TN (US)

(21) Appl. No.: 12/869,183

(22) Filed: Aug. 26, 2010

Related U.S. Application Data
(63) Continuation-in-part of application No. 11/697,933, filed on Apr. 9, 2007, now Pat. No. 7,799,234.
Application No. 11/697,933 is a continuation-in-part of application No. 11/697,921, filed on Apr. 9, 2007, now Pat. No. 7,794,606.

(51) Int. Cl. C02F 1/50; C02F 1/72

(52) U.S. Cl. 210/752; 210/192; 210/177; 210/173

(57) ABSTRACT

A waste treatment system and method for treating a substantially liquid waste stream. The waste treatment system includes a conditioning stage for conditioning the waste stream for treatment. A metal ion generation stage is provided for generating metal ions for disinfection of the waste stream and for catalytic oxidation. A wet oxidation stage is provided in fluid flow communication with the metal ion generation stage for denaturing the waste stream using an oxygen-containing gas. A chelation stage in fluid flow communication with the oxidation stage is provided for deactivating metal ions in the waste stream.

### CROSS-REFERENCE

This application is a continuation-in-part of application Ser. No. 11/697,933, filed Apr. 9, 2007, now allowed, and application Ser. No. 11/697,921, filed Apr. 9, 2007, now allowed.

### TECHNICAL FIELD

The disclosure relates to a waste and/or disinfection unit intended to provide for treatment and/or disinfection of liquid infectious wastes, including medical, domestic, scientific, mortuary, or commercial wastes, before flowing the waste stream to a sanitary sewer drain or directly to the environment.

### BACKGROUND AND SUMMARY

There is growing concern that biological infectious waste streams from hospitals, slaughter houses, and other sources that may contain biologically hazardous or toxic components are not adequately treated before discharging such waste streams to sanitary sewer systems or directly to the environment. Large municipal treatment facilities may not adequately be configured for high concentrations of biological materials originating in hospitals and other sources. Accordingly, there is a need for improved systems and methods for treating waste streams before the streams are discharged into a sanitary sewer system or directly to the environment. There is also a need for modular systems that may be readily deployed into an existing sanitary sewer system at the source of the waste stream thereby reducing the degree of infectivity of the material a municipal system must treat.

In view of the foregoing and other needs, an exemplary embodiment of the disclosure provides a waste treatment system for treating a substantially liquid waste stream. The waste treatment system includes a conditioning stage for conditioning the liquid waste stream for subsequent treatment. A metal ion generation stage is provided for generating metal ions for disinfection of the waste stream and for catalytic oxidation. A wet oxidation stage is in fluid flow communication with the metal ion generation stage for denaturing the waste stream using an oxygen-containing gas. A chelation stage in fluid flow communication with the oxidation stage is provided for deactivating metal ions in the waste stream.

Another exemplary embodiment of the disclosure provides a method of treating a waste stream. The method includes flowing the waste stream into a waste treatment apparatus; conditioning the waste stream for subsequent treatment; generating metal ions in the waste treatment apparatus for contact with the waste stream to disinfect the waste stream; generating metal ions in the waste treatment apparatus as a catalyst for oxidation treatment of the waste stream; oxidizing the waste stream in the waste treatment apparatus with an oxygen containing gas to eliminate any pharmaceutical compounds and biological activity in the waste stream; and chelating the waste stream in the waste treatment apparatus to deactivate any metal ions remaining in the waste stream.

An advantage of the system and methods described herein is that the system combines at least two disinfection techniques in a single treatment apparatus thereby increasing the effectiveness of waste stream disinfection over the use of a single disinfection technique. Unlike conventional systems, the active disinfection ingredients are deactivated prior to the waste stream being discharged from the disinfection unit so that the disinfection ingredients and waste stream may be discharged to the sanitary sewer system or directly to the environment without removing the disinfection ingredients from the waste stream. Because of the modular components of the system, the system may be configured as a mobile, or portable, stand-alone unit or may be provided in a substantially fixed non-portable installation that may be inserted between a waste material source and a final disposition of the waste material. The waste treatment system may also be combined and/or integral with a waste collection system or may be configured as a stand-alone system for discharge directly to the environment.

Additional features and advantages of the disclosure are set forth in part in the description which follows, and/or may be learned by practice of the disclosure. The features and advantages of the disclosure may also be realized and attained by means of the elements and combinations particularly pointed out in the appended claims.

It is to be understood that both the foregoing general description and the following detailed description are exemplary and explanatory only and are not restrictive of the disclosure, as claimed.

### BRIEF DESCRIPTION OF THE DRAWINGS

Further advantages of the exemplary embodiments may become apparent by reference to the detailed description of the exemplary embodiments when considered in conjunction with the following drawings illustrating one or more non-limiting aspects thereof, wherein like reference characters designate like or similar elements throughout the several drawings as follows:

FIG. 1 is a block flow diagram of one embodiment of a method of the present disclosure.

FIG. 2 is a schematic representation of one non-limiting example of an embodiment of a system of the present disclosure.

FIG. 3 is a schematic representation of another non-limiting example of an embodiment of a system of the present disclosure.

FIG. 4 is a block flow diagram of an embodiment of the disclosure for treating solid and liquid pharmaceutical waste materials according to the present disclosure.

FIG. 5 is a schematic representation of a modified system for treating liquid and solid pharmaceutical waste materials according to the present disclosure.

### DESCRIPTION OF THE EXEMPLARY EMBODIMENTS

As described in more detail below, embodiments of the present disclosure may provide systems and methods for disinfecting and/or detoxifying substantially liquid waste streams before discharging the waste streams to a sanitary sewer system or directly to the environment. The deactivation or destruction of infectious agents, such as viruses, bacteria, protists, fungi, algae, prions, or other infectious organic matter, through embodiments of the present disclosure may be herein referred to as “disinfection” or “biocidal activity.” The deactivation or destruction of toxic agents, such as pharmaceuticals, through embodiments of the present disclosure may be herein referred to a “detoxifying” or “detoxification.” The systems and methods may be adaptable to being portable or to being permanently attached to existing sanitary sewer drains. Each system may be substantially self-contained so that fluid discharged from the system may be suitable to flow into an existing sanitary sewer or directly to the environment without further disinfection or detoxification.

The systems and methods of the present disclosure may generate reactive agents in situ during the course of operation. Waste streams may be treated with a synergistic combination of metal ions and oxidants, such as hypochlorites, peroxides, or hydroxyl ions. However, it is not desirable to discharge metal ions and oxidants into the sewer system. Therefore, the waste streams may be further treated to ensure deactivation of the reactive agents and allow for discharge of the waste stream through a sewer system or directly to the environment.

Oxidants may be generated electrolytically in situ in the apparatus from water having a minimal conductivity. Likewise, metal ions may be generated in situ via redox reactions when electrodes comprising the appropriate metals are subjected to an electrical current of suitable polarity, voltage, and time duration. The ability to generate both metallic ions and reactive nonmetallic compounds in situ by electrolytic redox reactions provides an important technique for disinfection and detoxification of the waste stream on the spot as needed. Any active disinfecting agents not chemically deactivated as a result of the disinfection process may then be bound and deactivated by subsequent processing at the end of the disinfection cycle.

Unlike conventional disinfection systems, the system described herein may bind and deactivate the metal ions used for disinfection before discharge of a treated fluid stream to the sewer system, rather than removing the metal ions by filtration, precipitation, or other reaction or treatment mechanism. The presently disclosed system may charge a sufficient quantity of a chelating agent, such as EDTA or citric acid, to a chelation chamber, which may bind the metal ions and inactivate them. Chelation may also serve to bind unreacted oxidants that may be present in the stream. This binding technique is similar to the treatment used by physicians to treat ingestion of toxic metals for excretion from the body, and may allow for the chelated metals to be safely discharged into the sewer system or directly to the environment.

The present disclosure may also exploit the powerful biocidal properties of oxidizing agents, such as oxygen, hypochlorites, and peroxides, chlorine, ozone, and the like. The aforementioned and related chemicals, generally known as oxidants, are well known and established as highly effective microbicides. Such oxidants may be effective against viruses which may be either coated with an external protein coat, or uncoated. The oxidizing agents made and used in the system for their biocidal activity may also be deactivated or neutralized in situ by spontaneous reactions with organic compounds in the waste, without the need to remove these chemicals from the treated fluid stream.

While both metal ions and oxidizing agents are known to be effective biocidal agents individually, the combination of metal ions and oxidants in a single system may provide a synergistically improved effectiveness in biocidal activity that may be about 100,000 times greater than the disinfectant effectiveness of either the metal ions or the oxidants alone.

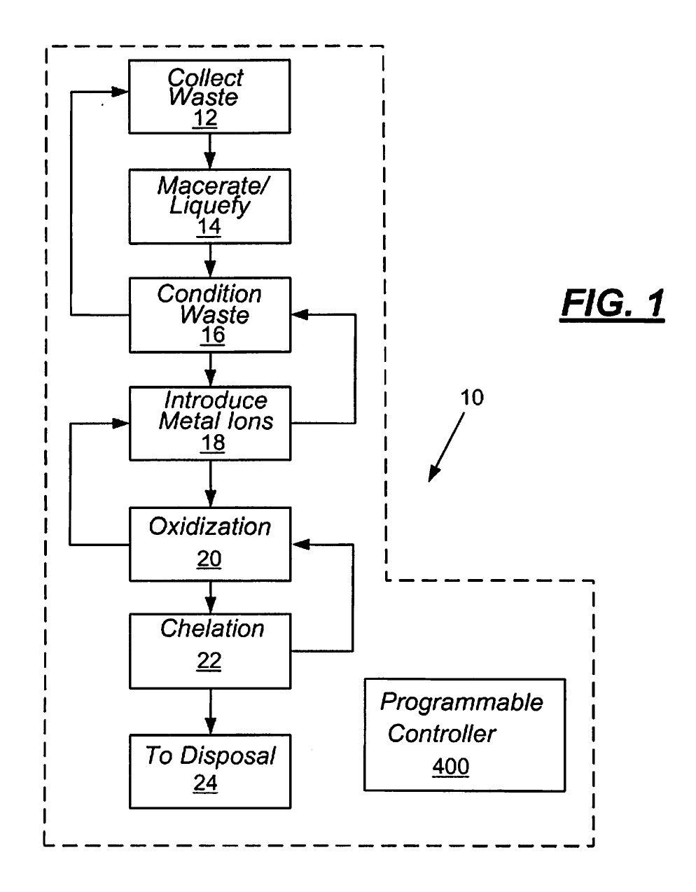

With reference to FIG. 1, one embodiment of a method of the present disclosure provides a system 10 that includes a series of continuous, in-line processes for the disinfection and/or detoxification of a substantially liquid waste stream, with each process stage further described in more detail below. Each process stage may herein be represented as an individual physical chamber or process stage in order to provide a clearer understanding of the in-line process concept. However, the present disclosure is not limited to individual chambers for each process stage, as in an alternative embodiment discussed below. The presently described process stages may be implemented either in individual chambers to simplify control or be combined in one or more common chambers to achieve an optimum footprint for the physical dimensions and cost/benefit to the unit design configuration.

A waste stream to be treated is first collected in a waste collection step 12, where water may be added if necessary to ensure that the waste material is substantially liquid or in substantially liquid form for further treatment. The viscosity of the waste stream may be adjusted with water in a conditioning stage 16 to provide a waste stream with a viscosity of from about 5 to about 200 centipoise at a temperature of from about 20° to about 25° C.

In order to generate metal ions in a reasonable amount of time for treatment of the waste stream in the system, a minimum conductivity of the waste stream is desirable. Accordingly, a saline solution such as a sodium chloride solution may be used to increase the conductivity of the waste stream in the conditioning stage 16. A suitable conductivity of the waste stream may be at least 300 microsiemens per centimeter at 25° C. A waste stream having a higher conductivity than 300 microsiemens per centimeter at 25° C. may be treated in the system 10, however, there is no particular advantage to providing too high a conductivity as it may be necessary to reduce the conductivity of the treated waste stream before disposal.

A maceration step 14 may be included prior to the conditioning stage 16. The maceration step 14 may ensure a homogenous particulate size for any organic matter that may be present in the substantially liquid waste material. The term “substantially liquid” means that any solids present in the waste material remain substantially suspended in a liquid phase for flow through the system 10 so that the solids as well as the liquid are treated in the system. If the particulate size of any organic matter present is too large for the waste to be processed in the next process step, the waste material may be recycled for further maceration in the maceration step 14.

A suitable particle size for solids in the waste stream after the maceration step may be less than about 0.5 millimeters in diameter and typically less than 0.3 millimeters in diameter after maceration. For example, the maximum particle may range from about 0.25 to about 0.5 millimeters in diameter. The initial particle size of particles in the waste stream entering the system 10 may range from about 5 to about 10 millimeters in diameter. The term “diameter” is used to signify an average cross-sectional dimension of particles based on the largest cross-section of the particles in the waste stream 112 and is not intended to indicate that the particles are necessarily circular or spherical.

In the conditioning step 16, in addition to adjusting the conductivity and/or viscosity of the waste stream, a component may be added to the waste stream to bind lipid complexes in the waste stream so as to prevent their agglomeration and adhesion of such lipid complexes to the walls of vessels and piping in the system. The component that may be added in the conditioning step 16 may be a film inhibitor such as fatty acid sulfate salt, e.g. sodium lauryl sulfate (“SLS”). The film inhibitor is believed to perform two critical functions. First, the film inhibitor may initiate a chemical attack to begin breaking down and denaturing both lipid and protein complexes present in the waste stream. Second, the film inhibitor's inherent detergency properties may enable the system 10 to remain “self-cleaning.” As an additional feature, SLS is also well known as a disinfecting and/or denaturing agent, and it may contribute to the overall synergistic disinfectant effect of the present system 10. Accordingly, SLS may be used to destroy noxious odor causing bacteria in the waste and/or prevent protein cascade which may cause the liquid phase waste stream to change from a free flowing fluid to a gelatinous mass which may inhibit further process treatment. An amount of SLS that may be metered into the system in the conditioning stage 16 may range from about 2.0 to about 10.0 percent by volume of the total volume of the waste stream.

Further conditioning of the waste stream may include contacting the waste stream with a surfactant in the conditioning stage 16. The surfactant may be used to prevent or inhibit the film inhibitor from forming a residual coating on the vessels and piping walls of the treatment system 10. A suitable surfactant may be a non-ionic surfactant, e.g. a polyether polyol surfactant. A particularly suitable non-ionic surfactant may have a hydrophilic/lipophilic balance (HLB) ranging from about 2 to about 8. The amount of surfactant used may vary depending on the amount of film inhibitor used. However, typically, the surfactant may be used in a concentration that ranges from about 1 to about 5 percent by volume based on the total volume of the waste stream.

Subsequent to the conditioning stage 16, metal ions may be introduced into the waste material in a metal ion generation stage 18, where electrolysis of a sacrificial electrode may generate oligodynamic concentrations of metal ions in situ. The waste stream may remain in or be recirculated through this stage 18 for a period of several minutes, in order to infuse an adequate concentration of metal ions into the waste stream and/or to allow sufficient time for the metal ions to at least partially disinfect the waste material.

The metal ion generation stage 18 may contain electrodes selected from two or more metals, including, but not limited to aluminum, silver, copper, iron, bismuth, gold, or zinc. The electrodes may be electrically connected to a power supply for generating metal ions in the waste stream corresponding to the metal composition of the electrodes. In an alternative embodiment, a metal ion generation chamber may itself be used as one of the electrodes. The conditioning stage 16 is provided prior to the metal ion generations stage 18 to condition the waste stream to have a conductivity that is conducive to the formation of metal ions in the waste stream.

Application of electrical energy to the electrodes may cause metal ions to be liberated from the electrodes via one or more redox reactions. The liberated metal ions may then become dissolved in the waste stream in the metal ion generation stage 18 so that the ions may provide disinfecting activity to the waste stream and catalytic activity for a subsequent oxidation stage 20. The voltage and current applied to the electrodes may be externally regulated in order to exercise control over the concentration of metal ions that may be dissolved in the waste stream in the metal ion generations stage 16.

Certain dissolved metal ions may act oligodynamically within the waste stream to deactivate or destroy bacterial, protist, fungal, algal, prion, and viral infectious agents present within the waste stream. In one embodiment, both silver and copper ions may be produced. It is believed that a concentration of copper ions that is much greater than a concentration of silver ions is particularly suitable for disinfection of waste liquids. For example, the copper ion concentration may range from about 1 to about 1000 ppm Cu ions by volume of the total volume of the waste stream and the silver ion concentration may range from about 0.5 to about 100 ppm Ag ions by volume of the total volume of the waste stream. A concentration ratio of from about 5:1 to about 10:1 Cu ions to Ag ion may be a highly effective ratio of copper ions to silver ions in the metal ion generation stage 18. Other ratios of copper to silver ions may also be suitable for treatment of the waste stream. In one embodiment, a suitable copper ion concentration may range from about 100 ppm to about 1000 ppm by volume, with a further suitable example being about 400 ppm by volume of copper ions. Likewise, suitable silver ion concentration may range from about 10 ppm to about 100 ppm by volume, with a further suitable example being about 40 ppm by volume of silver ions. A suitable total metal ion concentration for disinfection may range from about 110 ppm to about 1100 ppm by volume. As a further example, a suitable total metal ion concentration may range from about 200 ppm to about 800 ppm volume, and as another suitable example a total metal ion concentration may range from about 300 ppm to about 600 ppm by volume. Furthermore, ions of different metals may be produced at different concentration levels in order to provide a suitable total dissolved metal ion concentration in the waste stream.

In order to facilitate or catalyze wet oxidation of the waste stream in an oxidation stage 20, iron ions may also be generated in the metal ion generation stage 18. Iron electrodes may also be present in the metal ion generation stage 18 and be energized to provide a suitable level of iron ions in the waste stream. Iron ions in an amount ranging from about 10 to about 1000 ppm by volume based on the total volume of the waste stream may be suitable for effective catalytic oxidation of components in the waste stream.

A metal ion exposure time ranging from about 1 to about 30 minutes may be suitable to provide disinfection to the waste stream, with a further suitable example ranging from about 5 to about 10 minutes of exposure time. Particularly resistant wastes may require additional time or higher concentration of the metal ions. Variations in operation may be accommodated by process control using a programmable controller as part of the system 100.

The electrodes used to produce the metal ions may be pure metals in which multiple pairs of electrodes may be used and voltages and currents to each electrode pair regulated independently in order to control the various metal ion concentrations. The electrodes may also be composed of a mixture of more than one metal, such as a metal alloy, in order to control the concentration of each ion in solution. Each of the electrodes in the pair of electrodes may comprise a distinct and independent composition.

The electrodes may be fabricated employing powder metallurgy. A further embodiment using copper powder and silver or silver-alloy “solder” may be employed as a binder. The powdered metal electrodes may be fabricated such that the concentration of exposed metals such as copper or silver could be carefully controlled to produce the desired concentration ratio of metal ions. Additionally, the composition of each electrode and its corresponding ionic contribution may be controlled through particle size and amount of each phase, primary metal and “binder” present in the powder molded electrode. For example, large spherical grains of copper may be pressed with silver solder powder and sintered to form an electrode with higher surface concentrations of copper. Studies have shown that a combination of copper and silver ions wherein the concentration of copper ions is much higher than the concentration of silver ions may be very effective in disinfecting liquid containing biological hazards.

In a further embodiment of the present disclosure, one or more electrodes may be integrated into a mixing device or mixing pump for the metal ion generation stage 18 in which the various vanes or other portions of the mixing device may also act as an electrode. In an alternative embodiment, a mixing device may comprise an electrode and may be used in combination with other electrodes.

Periodically, the current on the electrodes, particularly positive electrodes, for generation of the metal ions may be reversed in order to clean the surface of the electrodes. For example, the lipid complexes in the waste stream may bond to the positive electrodes during metal ion generation. Reversing the polarity on the positive electrodes may cause bio-films formed from the lipid complexes to be driven off of the electrodes. Accordingly, during the ion generation step, the polarity of the electrodes may be alternately switched from positive to negative, or after each ion generation step an electrode cleaning cycle may be used to switch the polarity on the electrodes. The polarity on the electrodes may be switched for a period of time that is less than a time required for generating metal ions in the waste stream. In order to clean the electrodes of bio-films that may have been formed on the electrodes. For example, if the time for generating metal ion ranges from about 3 to about 5 seconds per minute, the polarity may be reversed for a period of 1 to 2 seconds per minute. During a system cleaning cycle, in addition to reversing the polarity on the electrodes, the electrodes may be flushed with clean water to remove bio-films from the electrodes.

In the next step of the process, the waste material may be oxidized in an oxidation stage 20. The oxidation stage 20 may be a wet oxidation stage 20 using an oxygen containing gas. The wet oxidation stage may be enhanced by heating the waste stream containing metal ions from the metal ion generation stage 18 to a temperature ranging from about 30° to about 90° C. According to embodiments of the disclosure, pure oxygen is the most desirable oxygen-containing gas for conducting the oxidation stage 20 of the process 10 because pure oxygen substantially reduces a gas volume requirement for treating the waste stream while providing a maximum amount of oxygen. Based upon the readings from an oxygen demand sensor, subsequent to the oxidation stage 20, the waste stream may be recycled or may be suitable for discharge to a sanitary sewer system.

During the oxidation stage 20 of the process 10, the chemical oxygen demand (COD) and/or biological oxygen demand (BOD) of oxidized liquid formed during the oxidation stage 20 may be monitored with sensors, such as an oxygen demand sensor, to determine the amount of oxygen required to treat all of the incoming waste material. A target resulting COD or BOD for the treated waste material is in a range defined by regulatory requirements at a user's location.

According to the disclosure, oxidation of the waste stream takes place in an aqueous environment wherein water is an integral part of the reaction. Water provides a medium for dissolved oxygen to react with organics and other oxidizable materials in the waste stream. It is believed that wet oxidation involves free radical formation with oxygen derived radicals attacking the organic compounds and resulting in the formation of organic radicals.

A noteworthy characteristic of wet oxidation chemistry is the formation of carboxylic acids in addition to CO₂ and water. Other oxidation products as a result of treating the waste stream in the oxidation stage 20, may include, but are not limited to sulfur dioxide, nitrogen dioxide, and phosphorus pentoxide which may be dissolved in the oxidized liquid. The yield of carboxylic acids varies greatly depending on the design of the system and may formed with about 5 to about 10 weight percent of the total organic carbon (TOC) in the waste stream. The primary carboxylic acids formed as a result of wet oxidation include acetic acid, formic acid, and oxalic acid. Such carboxylic acids are typically biodegradable and conventional biological post treatment of the oxidized waste stream may be conducted to reduce the amount of acids in the waste stream exiting the oxidation stage 20.

Subsequent to the oxidation stage, a chelation stage 22 may be used to post-condition the waste stream by binding any free metal ions that may remain in the waste stream after the oxidation stage 20 with a chelating agent. The waste stream may be treated in this stage for a period of several minutes, in order to allow sufficient time for the chelating agent to sequester substantially all of the metal ions. Chelation may also serve to bind unreacted oxidants that may be present in the waste stream. Subsequent to the chelation stage 22, the waste stream may be discharged or disposed of in a waste disposal stage 24 to a sanitary sewer system or directly to the environment. The waste disposal stage 24 may be used to confirm that the waste stream has been fully treated and that substantially no metal ions remain in the waste stream

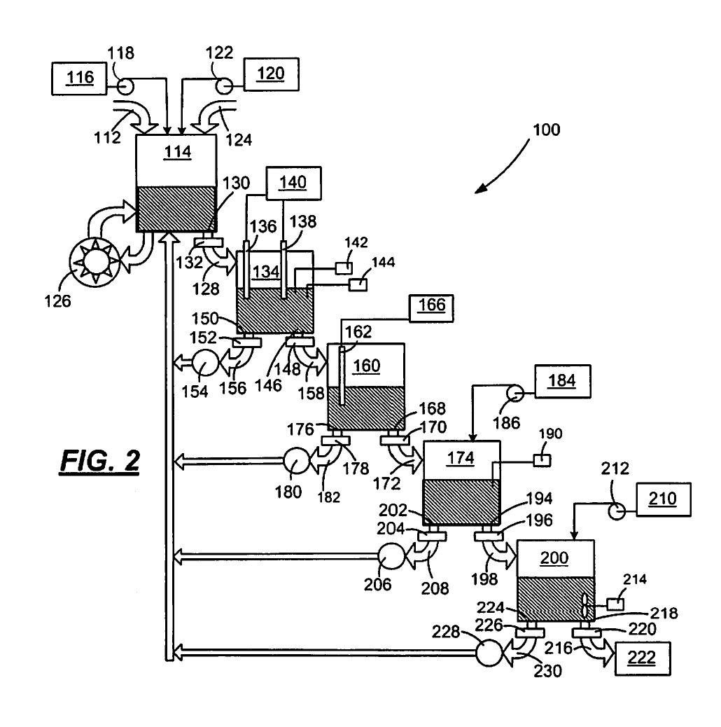

In order to further illustrate aspects of the disclosed embodiments, reference is made to FIG. 2. FIG. 2 is a schematic illustration of a multi-vessel system 100 for treatment of a waste stream 112. In the multi-vessel system 100, one or more stages of the process 10 may be conducted in one or more of the vessels. For example, the waste collection stage 12, maceration stage 14 and conditioning stage 16 may be conducted in conjunction with a collection/conditioning vessel 114.

As shown in FIG. 2, the waste stream 112 comprising biological waste and solids may be fed into collection/conditioning vessel 114. A film inhibitor reservoir 116 including a film inhibitor dosing pump 118, and a surfactant reservoir 120 including a surfactant dosing pump 122 may be associated with the collection/conditioning vessel 114 for feeding a film inhibitor and/or surfactant into the collection/conditioning vessel 114. A saline solution and/or water may be introduced to the collection/conditioning vessel 114, as needed, via an additional input line 124 to provide the waste stream in the collection/conditioning vessel 114 with a suitable viscosity and conductivity for treatment as set forth above.

A macerator 126 may also be associated with the collection/conditioning vessel 114, wherein the waste material in the collection/conditioning vessel 114 may be mechanically macerated to reduce the size of any solid material in the waste steam and collection/conditioning vessel 114. One or more macerators 126 may be used for chopping or mixing material within the vessel 114 in order to macerate and mix the incoming waste stream 112 with the water and the solution of film inhibitor, surfactant, and saline solution. Macerating the waste stream 112 may also be effective to extend the time of contact between the waste stream 112 and metal ions or oxidizing chemicals in the system 100 for any portion of the waste stream that may be recycled to the collection/conditioning vessel 114. Maceration may aid to break down organic solids in the waste stream 112 to provide a rough particle size as set forth above and to provide a homogenized waste stream 128 for more effective treatment in the system 100.

The collection/conditioning vessel 114 may be constructed of copper or copper alloy to provide an inherent bactericidal action thereby suppressing undersirable bacterial growth. Similar bactericidal action may be obtained by copper plating or use of a copper or copper alloy floorplate in the collection/conditioning vessel 114.

If desired a foam suppressant, such as a silicone-based antifoam agent, may also be introduced into the collection/conditioning vessel 114 to ensure that air bubble formation is reduced during maceration cycles, so that air entrapment does not inhibit the operating efficacy of the reactive disinfectant species generated in the subsequent stages of the process 10. An amount of foam suppressant that may be used to suppress air entrapment in the vessel 114 may range from about 0.05 to about 1.0 percent by volume.

The collection/conditioning vessel 114 may further comprise a fluid exit port 130 comprising a unidirectional valve 132 and/or a pump for allowing the homogenized waste stream 128 to enter a metal ion generation vessel 134 that may be in fluid flow communication with the collection/conditioning vessel 114.

The metal ion generation vessel 134 contains at least one pair of electrodes 136, 138 that may be electrically connected to a power supply 140. The electrodes 136, 138 may comprise one or more metals, selected from aluminum, silver, copper, iron, bismuth, gold, or zinc. The metal composition of the electrodes 136, 138 may provide a source for the electrolytic generation of corresponding metal ions during the metal ion generation stage 18.

The power supply 140 may provide electrical energy for the electrodes 136, 138. Application of electrical energy to the electrodes 136, 138 may cause metal ions to be liberated from the electrodes into the waste stream via one or more redox reactions. The liberated metal ions may then become dissolved in the waste stream in the metal ion generation vessel 134 so that the ions may provide disinfecting activity to the waste stream. The voltage and current applied to the electrodes 136, 138 may be externally regulated in order to exercise control over the concentration of metal ions that may be dissolved in the waste stream in the vessel 134.

Multiple pairs of electrodes 136, 138, each electrode comprising one or more metal compositions and having an appropriate voltage and current flow, may be used to introduce various concentrations of one or more metal ions into the waste stream. As set forth above, the metal ion generation vessel 134 may itself be used as one of the electrodes. In one embodiment, the electrodes include silver, copper electrodes 136, 138.

The metal ion generation vessel 134 may further comprise a fluid level meter 142 for controlling a level of fluid in the vessel 134 and a conductivity meter 144 for controlling the amount of saline solution 124 added in the collection/conditioning vessel 114. The metal ion generation vessel 134 may also comprise a fluid exit port 146 comprising a directionally restrictive fluid flow valve 148 and/or pump for flow into a subsequent treatment vessel.

A second fluid exit port 150 comprising a recycling valve 152 and/or pump 154 may allow at least a portion 156 of the waste treated in the metal ion generation vessel 134 to be returned to the collection/conditioning vessel 114 so that the portion 156 of waste may be recycled through the system 100 and further disinfected.

The metal ion treated waste stream 158 may then be passed into an iron ion generation vessel 160. The iron ion generation vessel 160 may include an iron electrode 162 for generating iron ions in the waste material in the vessel 160. The iron electrode 162 may be in electrical connection with a power supply 166. Application of electric current to the electrode 162 may generate iron ions to be dissolved in the waste material for catalyzing a subsequent oxidation stage of the process 10. A suitable concentration of iron ions in the waste material is described above. In an alternative embodiment, the iron electrode 162 may be included with the copper and silver electrodes 136 and 138 in the metal ion generation vessel 134.

The waste material containing iron ions may then flow through exit port 168 and valve 170 as an iron ion containing waste steam 172 into an oxidation vessel 174 for wet oxidation of the waste stream 172. A heat exchanger may be provided for heating the waste stream 172 entering the oxidation vessel 174.

A second fluid exit port 176 comprising a recycling valve 178 and/or pump 180 may allow at least a portion 182 of the waste material from vessel 160 to be passed to the collection/conditioning vessel 114 or to a previous vessel 134 so that the portion 182 of waste material may be recycled through the system 100 and further treated.

An oxygen-containing gas from an oxygen supply 184 may be provided by a compressor 186 to the oxidation vessel 174. The oxygen-containing gas may be sparged into the oxidation vessel 174 below a liquid level in the vessel 174 and/or the waste stream 172 may be sprayed through a spray mixing nozzle with the oxygen-containing gas into the oxidation vessel 174 for intimate contact with the oxygen containing gas. The oxidation vessel 174 may be further pressurized by the compressor 186 to provide a chamber operating pressure in the range of from about 0.01 to about 0.2 MPa above ambient pressure. Removal of copper and silver ions from the waste material 172 prior to flow of the waste material 172 into the oxidation vessel 174 may not necessary as the metal ions, in combination with the iron ions may further aid in the oxidation treatment step of the process.

In the oxidation vessel 174, the chemical oxygen demand (COD) and/or biological oxygen demand (BOD) of oxidized liquid formed in the oxidation vessel 174 may be monitored with sensors, such as an oxygen demand sensor 190 to determine the amount of oxygen required to treat all of the incoming waste material 172. A target resulting COD or BOD for oxidized waste material is in a range defined by regulatory requirements at a user's location.

According to the disclosure, oxidation of the waste material takes place in an aqueous environment wherein water is an integral part of the reaction. Water provides a medium for dissolved oxygen to react with organics and other oxidizable materials in the oxidized waste material. It is believed that wet oxidation involves free radical formation with oxygen derived radicals attacking the organic compounds and resulting in the formation of organic radicals.

The oxidized waste material may then be passed through an exit port 194 and exit valve 196 as a treated stream 198 into a chelation vessel 200 for treatment and removal of metal ions from the treated stream 198. A second exit port 202, recycle valve 204 and recycle pump 206, may be provided to pump a portion 208 of the treated stream into the collection/conditioning vessel 114 or any one or more of the previous vessels 134 and 160 for further treatment and/or for assisting in control of the treatment system 100. A quantity of chelating agent may be provided to the waste material in the chelation vessel 200 from a reservoir 210 by the dosing pump 212 in order to facilitate removal of the metal ions. A mixing device 214 may provide continuous circulation and contact of the waste with the chelation agent in the chelating vessel 200. The waste material in the chelation vessel 200 may be processed in a timed chelation cycle that may allow the metal ions to be bound chemically to the chelating agent and may ensure that the oxidants have fully reacted with any organic materials present in the waste material.

The timed chelation cycle may range in duration from about 1 to about 30 minutes, with another suitable example being from about 5 to about 10 minutes. The amount of chelating agent in the chelation vessel 200 sufficient to chemically bind any metal ions may range from about a molar equivalent of the metal ion concentration to about one and a half times a molar equivalent of the metal ion concentration. Typically, the amount of chelating agent will be about a molar equivalent of the metal ion concentration in the treated stream 198 entering the chelating vessel 200. A suitable chelating agent may be selected from EDTA, citric acid, sodium citrate, acetylacetone, ethylenediamine, diethylenetriamine, tetramethylethylene-diamine, 1,2-ethanediol, 2,3-dimercaptopropanol, porphyrin, gluconic acid, or similar compounds.

Following completion of the timed chelation cycle, a treated waste stream 216 may be discharged through a fluid exit port 218, a unidirectional valve 220 and/or pump, into a sanitary sewer system 222 in a disinfected and chemically inert state by either pumping or gravity flow into a sanitary sewer drain. As with the other vessels, described above, the chelation vessel 200 may further comprise a second fluid exit port 224 comprising a recycling valve 226 and/or pump 228 that may allow at least a portion 230 of the waste material to be returned to the collection/conditioning vessel 114 or a previous vessel so that the portion of waste 230 may be recycled through the system 100 and further disinfected.

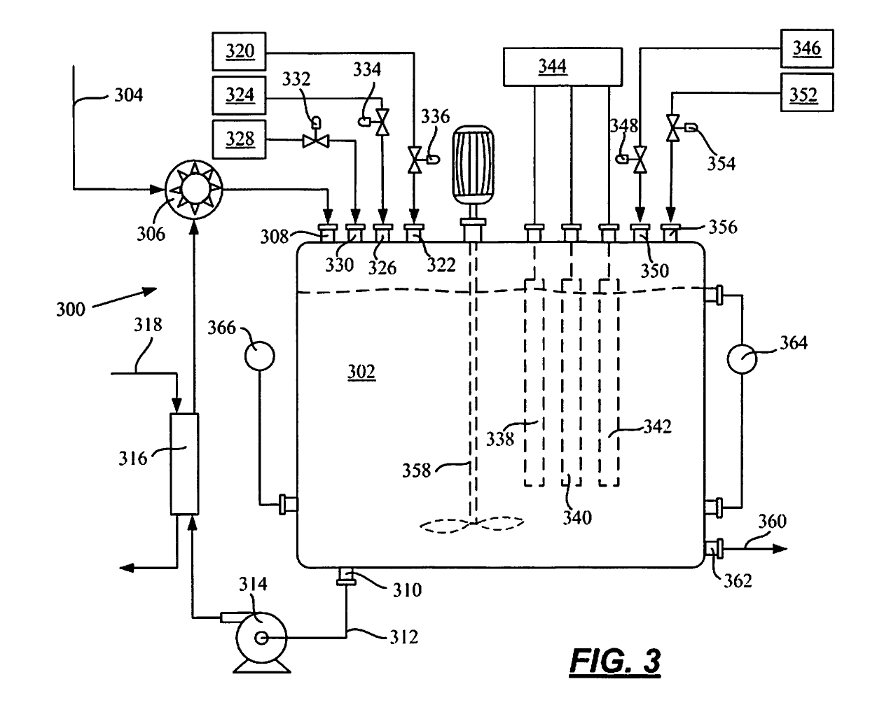

As described above, one or more of the vessels 114, 134, 160, 174, and/or 200 may be combined to provide a multifunctional vessel for a treatment system 300 according to the disclosure. Such a multifunctional vessel 302 for a treatment system 300 according to the disclosure is illustrated schematically in FIG. 3.

A waste stream 304 to be treated may be flowed through a macerator 306 for reducing the particle size of any solid materials that may be in the waste stream 304. A waste stream inlet port 308 may be provided on the vessel 302 for introducing the waste stream into the vessel 302. A recycle port 310 may be provided on the vessel 302 for recycling a portion 312 of the waste stream via a pump 314 to the macerator 306. In order to enhance the effectiveness of the oxidation stage 20 of the process 10, a heat exchanger 316 may be provided for heating or cooling the portion 312 of the waste stream with a heating or cooling fluid 318.

As described above with respect to the system 100, a supply of film inhibitor 320 may be provided through an inlet port 322 into the vessel 302. A surfactant 324 may be provided through inlet port 324, and a saline solution 328 may be provided through inlet port 330. In order to reduce the number of inlet ports on the vessel 302, one or more of the foregoing components may share an inlet port by means of isolation valves 332, 334, and 336.

Electrodes 338, 340, and 342 may be inserted in the vessel 302 to provide metal ions, as described above, for the ion generation stage 18 and for the catalytic oxidation stage 20. In the embodiment illustrated, the electrodes include a copper ion generation electrode 338, a silver ion generation electrode 340, and an iron ion generation electrode 342. Power to the electrodes 338, 340, and 342 is provided by a power source 344.

During the oxidation stage 20, an oxygen-containing gas from an oxygen source 346 is provided through a control valve 348 and inlet port 350 to the vessel 302. After completion of the oxidation stage 20, a chelation agent is provided from a supply 352 through a control valve 354 and inlet port 356 to the vessel 302. A mixing device 358 may be provided to assure adequate contact between the waste material in the vessel 302 and one or more of the conditioning agents, metal ions, oxidation agent, and chelation agent so that the treated material 360 exiting the vessel 302 through exit port 362 may be disposed in a sewer system without further treatment. Monitoring devices such as a fluid level control 364 and a conductivity probe 366 may be provided to further monitor and control the treatment system 300.

Embodiments of the present disclosure may also comprise a programmable controller 400 (FIG. 1) capable of interfacing with the fluid level control 364, conductivity probe 366, and other sensors that may be present in order to coordinate the activities of dosing pumps, control valves 332, 334, 336, 348, 354, heat exchanger 316, macerator 306, power source 344, and the like during the treatment process 10. The controller 400 may be used to estimate the amount of waste being processed, to control the conductivity of the waste material, to control the temperature of the material being treated, to control the electrode voltage and currents responsible for producing the active disinfecting agents, to control the time intervals for each stage of waste processing, to maintain a suitable level of fluid in the vessel 302. The controller 230 may also estimate the amount of chelating agent required during the chelation stage 22 of the process.

Variations between the multiple vessel system 100 and the single vessel system 300 may be used to provide a compact and/or portable system. The system 100 may be arranged in such a manner so as to reduce space requirements for the various process stages. Likewise, the system 300 may be designed to provide a compact single vessel for performing multiple stages of the process. Other arrangements of the components of the system may be possible so that a reduced height and a reduced length the systems 100 and/or 300 are provided.

The systems 100 and 300 may be particularly adapted to treating waste liquid streams containing bacteria, surgical waste, biological or biologically toxic materials, solids, pharmaceuticals, and the like. Such materials may include, but are not limited to, dairy shed waste, fowl waste, milk processing plant waste, food processing wastes, waste from the wine and brewery industries, food waste, shipboard waste, sewage, medical waste, and the like.

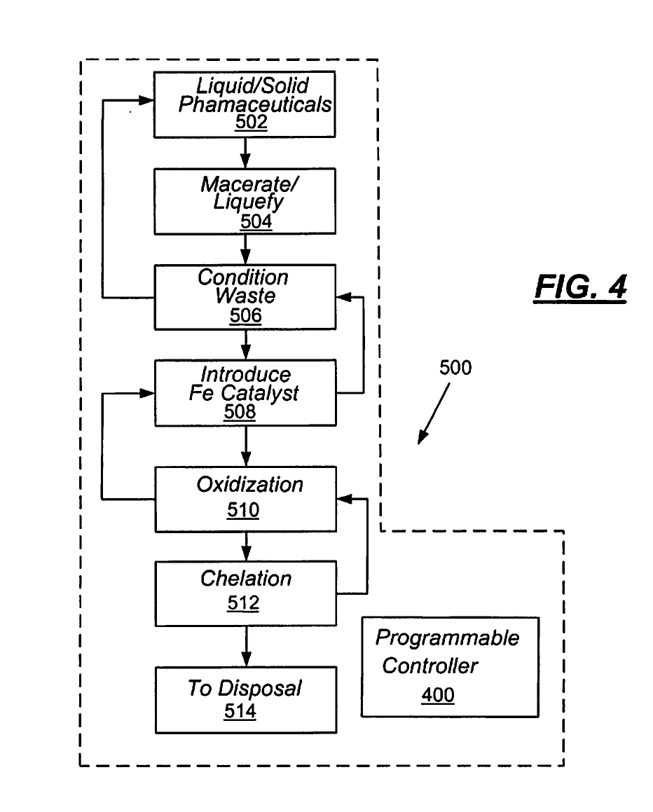

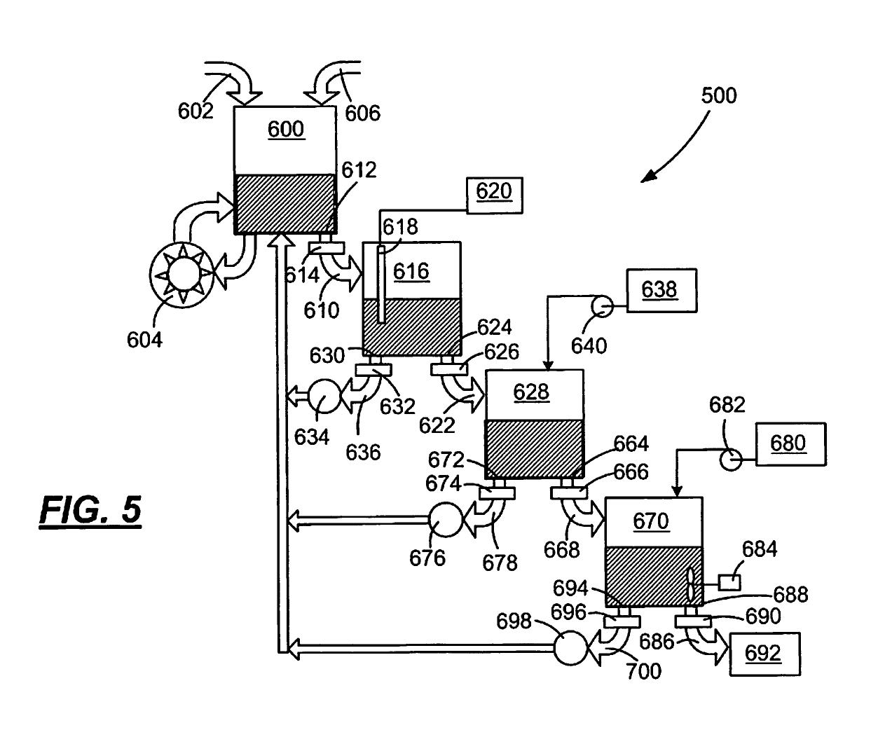

A system 500 specifically adapted for treating liquid and solid pharmaceutical waste materials is illustrated in FIGS. 4-5. Pharmaceutical waste materials may include, but are not limited to, capsules—solid, liquid, powder, solid tablets/pills, gels, liquids/syrups/suspensions, injectables, mists, creams/ointments, patches, and the like. Such waste materials include both prescription pharmaceutical waste materials and over the counter pharmaceutical waste materials.

Accordingly, a flow diagram for the system 500 may only include the process steps of collecting the liquid or solid pharmaceutical materials (Step 502), macerating or otherwise grinding the materials in Step 504 to provide a liquefied waste stream for flow through the system 500, conditioning the waste stream in Step 506 to adjust the viscosity, introducing iron ions into the waste stream in Step 508 as a catalyst for a subsequent oxidation step, oxidizing the waste stream in Step 510, and removing any residual metal ions in Step 512 prior to disposal of the waste stream (Step 514). The foregoing process steps may be controlled by the same programmable controller 400.

As in system 100, a suitable particle size for solids in the waste stream after the maceration step 504 may be less than about 0.5 millimeters in diameter and typically less than 0.3 millimeters in diameter after maceration. For example, the maximum particle may range from about 0.25 to about 0.5 millimeters in diameter. The term “diameter” is used to signify an average cross-sectional dimension of particles based on the largest cross-section of the particles in the waste stream and is not intended to indicate that the particles are necessarily circular or spherical.

The conditioning step 506 of the process 500 may take place in vessel 600 wherein solids in the waste stream 602 are reduced in size using a macerator 604, and wherein the viscosity of the waste stream 602 may be adjusted to provide a waste stream with a viscosity of from about 5 to about 200 centipoise at a temperature of from about 20° to about 25° C. As in system 100, described above, a saline solution and/or water may be introduced to the collection/conditioning vessel 600, as needed, via an input line 606 to provide the waste stream in the collection/conditioning vessel 600 with a suitable viscosity and conductivity for further treatment.

Since the pharmaceutical waste material may be substantially devoid of infectious biological waste materials, it may not be necessary to use the entire treatment cycle described above with reference to system 100. For example, the film inhibitor and surfactant used in system 100 may not be necessary since the pharmaceutical waste stream is not likely to contain biological materials that may stick to the equipment. Also, only a single metal ion species, such as iron ions may be generated in order to catalyze the oxidation step 510 of the process 500.

Accordingly, after the conditioning step 506, the conditioned pharmaceutical waste stream 610 may exit vessel 600 through exit port 612 and exit valve 614 and flow into vessel 616 wherein iron catalyst ions are introduced into the waste stream in step 508. Vessel 616 may thus include an iron electrode 618 and a power source 620 for providing iron ions in an amount ranging from about 10 to about 1000 ppm by volume based on the total volume of the waste stream.

The waste material 622 containing iron ions may then flow through exit port 624 and valve 626 into an oxidation vessel 628 for wet oxidation of the waste stream 622. A heat exchanger may be provided for heating the waste stream 622 entering the oxidation vessel 628.

As in the system 100, a second fluid exit port 630 comprising a recycling valve 632 and/or pump 634 may allow at least a portion 636 of the waste material from vessel 616 to be passed to the collection/conditioning vessel 600 so that the portion 636 of waste material may be recycled through the system 500 for process control or further treatment.

An oxygen-containing gas from an oxygen supply 638 may be provided by a compressor 640 to the oxidation vessel 628. The oxygen-containing gas may be sparged into the oxidation vessel 628 below a liquid level in the vessel 628 and/or the waste stream 622 may be sprayed through a spray mixing nozzle with the oxygen-containing gas into the oxidation vessel 628 for intimate contact with the oxygen containing gas. The oxidation vessel 628 may be further pressurized by the compressor 640 to provide a chamber operating pressure in the range of from about 0.01 to about 0.2 MPa above ambient pressure. In the oxidation vessel 628, the pharmaceutical material is oxidized or otherwise rendered inert so that it may be discharged from the system 500 without further treatment. It is believed that wet oxidation, as described herein, involves free radical formation with oxygen derived radicals attacking organic compounds that may be present in the waste material resulting in the formation of organic radicals.

The oxidized waste material may then be passed though an exit port 664 and exit valve 666 as a treated stream 668 into a chelation vessel 670 for treatment and removal of metal ions from the treated stream 668. A second exit port 672, recycle valve 674 and recycle pump 676, may be provided to pump a portion 678 of the treated stream into the collection/conditioning vessel 600 or the vessel 616 for further treatment and/or for assisting in control of the treatment system 500. A quantity of chelating agent may be provided to the waste material in the chelation vessel 6700 from a reservoir 680 by the dosing pump 682 in order to facilitate removal of any remaining iron ions. A mixing device 684 may provide continuous circulation and contact of the waste with the chelation agent in the chelating vessel 670. As in system 100, the waste material in the chelation vessel 670 may be processed in a timed chelation cycle that may allow the iron ions to be bound chemically to the chelating agent and may ensure that the oxidants have fully reacted with any organic materials present in the waste material.

The timed chelation cycle may range in duration from about 1 to about 30 minutes, with another suitable example being from about 5 to about 10 minutes. The amount of chelating agent in the chelation vessel 670 sufficient to chemically bind any iron ions may range from about a molar equivalent of the iron ion concentration to about one and a half times a molar equivalent of the iron ion concentration. Typically, the amount of chelating agent will be about a molar equivalent of the iron ion concentration in the treated stream 668 entering the chelating vessel 670. A suitable chelating agent may be selected from EDTA, citric acid, sodium citrate, acetylacetone, ethylenediamine, diethylenetriamine, tetramethylethylene-diamine, 1,2-ethanediol, 2,3-dimercaptopropanol, porphyrin, gluconic acid, or similar compounds.

Following completion of the timed chelation cycle, a treated waste stream 686 may be discharged through a fluid exit port 688, a unidirectional valve 690 and/or pump, into a sanitary sewer system 692 in a chemically inert state by either pumping or gravity flow into a sanitary sewer drain. As with the other vessels, described above, the chelation vessel 670 may further comprise a second fluid exit port 694 comprising a recycling valve 696 and/or pump 698 that may allow at least a portion 700 of the waste material to be returned to the collection/conditioning vessel 600 or a previous vessel so that the portion of waste 700 may be recycled through the system 500 and further treated.

The programmable controller 400 may be programmed to provide treatment of waste streams according to system 100 and/or system 500 by including only certain steps in the treatment process. As described above, variations between the multiple vessel system 500 and the single vessel system 300 may be used to provide a compact and/or portable system. The system 500 may be arranged in such a manner so as to reduce space requirements for the various process stages. Other arrangements of the components of the system may be possible so that a reduced height and a reduced length of the system are provided.

In embodiments of the present disclosure, replaceable electrodes and replaceable cartridges or refillable reservoirs of chelating agents, film inhibitors, foam suppressants, surfactants, and saline solution may be provided. In one embodiment, the cartridges or reservoirs and the electrodes are accessible from the exterior of the system in order to facilitate ease of refilling or replacement by the user.

As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Other embodiments of the present disclosure will be apparent to those skilled in the art from consideration of the specification and practice of the embodiments disclosed herein. Accordingly, the embodiments are not intended to be limited to the specific exemplifications set forth hereinabove. Rather, the foregoing embodiments are within the spirit and scope of the appended claims, including the equivalents thereof available as a matter of law.

The patentees do not intend to dedicate any disclosed embodiments to the public, and to the extent any disclosed modifications or alterations may not literally fall within the scope of the claims, they are considered to be part hereof under the doctrine of equivalents.

What is claimed is:

1. A waste treatment system for treating a substantially liquid waste stream, comprising:
a conditioning stage for conditioning the liquid waste stream for subsequent treatment;
a metal ion generation stage for generating metal ions for disinfection of the waste stream and for catalytic oxidation;
a wet oxidation stage in fluid flow communication with the metal ion generation stage for denaturing the waste stream using an oxygen-containing gas; and
a chelation stage in fluid flow communication with the oxidation stage for deactivating metal ions in the waste stream.

2. The waste treatment system of claim 1, wherein two or more of the conditioning stage, the metal ion generation stage, the wet oxidation stage and the chelation stage comprise a single combined chamber for conducting each stage.

3. The waste treatment system of claim 1, wherein the metal ion generation stage comprises at least one metal ion generating electrode for generation of one or more metal ions in situ, and wherein the at least one metal ion generating electrode comprises one or more metals selected from the group consisting of copper, aluminum, zinc, iron, bismuth, gold, and silver.

4. The waste treatment system of claim 1, wherein the wet oxidation stage comprises an oxygen-containing gas introduced into a waste stream that is heated to a temperature ranging from about 30° to about 90° C.

5. The waste treatment system of claim 1, further comprising a maceration stage prior to the conditioning stage, the maceration stage comprising a maceration device for providing reduced particle size of material in the waste stream.

6. The waste treatment system of claim 1, wherein the conditioning stage provides the liquid waste stream with a conductivity of at least 300 microsiemens per centimeter at 25° C.

7. The waste treatment system of claim 1, wherein the conditioning stage comprises one or more of a viscosity adjustment port, a film inhibitor port, a conductivity adjustment port, a viscosity adjustment port, and a surfactant port.

8. The waste treatment system of claim 1, wherein the metal ion generation stage provides the waste stream with a copper ion concentration raging from about 1 to about 1000 ppm by weight and a silver ion concentration ranging from about 0.5 to about 100 ppm by weight based on a total weight of the waste stream.

9. The waste treatment system of claim 8, wherein the metal ion generation stage provides the waste stream with an iron ion concentration ranging from about 10 to about 1000 ppm by volume based on a total volume of the waste stream.

10. The waste treatment system of claim 9, wherein copper, silver and iron ions are generated in a single chamber.

11. A method of treating a waste stream comprising:
flowing the waste stream into a waste treatment apparatus;
conditioning the waste stream for subsequent treatment;
generating metal ions in the waste treatment apparatus for contact with the waste stream to disinfect the waste stream;
generating metal ions in the waste treatment apparatus as a catalyst for oxidation treatment of the waste stream;
oxidizing the waste stream in the waste treatment apparatus with an oxygen containing gas to eliminate any pharmaceutical compounds and biological activity in the waste stream; and
chelating the waste stream in the waste treatment apparatus to deactivate any metal ions remaining in the waste stream.

12. The method of claim 11, wherein the metal ions comprise metal ions selected from the group consisting of aluminum, zinc, silver, iron, bismuth, gold, and copper ions.

13. The method of claim 11, wherein the oxidizing step comprises exposing the waste stream to an oxygen containing gas wherein the waste stream is at a temperature ranging from about 30° to about 90° C.

14. The method of claim 11, wherein the chelating step comprises exposing the waste stream to one or more chelating compounds selected from the group consisting of EDTA, citric acid, sodium citrate, acetylacetone, ethylenediamine, diethylenetriamine, tetramethylethylenediamine, 1,2-ethanediol, 2,3-dimercaptopropanol, porphyrin, and gluconic acid.

15. The method of claim 11, further comprising macerating the waste stream prior to the conditioning step to reduce a particle size of material in the waste stream to form a substantially liquid flowable waste material.

16. The method of claim 11, wherein the waste stream is conditioned in the conditioning step to provide the waste stream with a viscosity ranging from about 5 to about 200 centipoise at 20° C. and a conductivity of at least about 300 microsiemens per centimeter at 25° C.

17. The method of claim 11, further comprising treating the waste stream in the conditioning step with an amount of a compound effective for inhibiting a film forming tendency of the waste stream.

18. The method of claim 11, wherein the metal ion generating step provides the waste stream with a copper ion concentration raging from about 1 to about 1000 ppm by weight and a silver ion concentration ranging from about 0.5 to about 100 ppm by weight based on a total weight of the waste stream.

19. The method of claim 18, wherein the metal ion generating step provides the waste stream with an iron ion concentration ranging from about 10 to about 1000 ppm by volume based on a total volume of the waste stream.

20. The method of claim 11, wherein one or more of the metal ion generation steps, oxidation step, and chelating step are conducted in a single vessel.

21. A method of treating a pharmaceutical waste stream comprising:
flowing the waste stream into a waste treatment apparatus;
conditioning the waste stream for subsequent treatment;
generating iron ions in the waste treatment apparatus as a catalyst for oxidation treatment of the waste stream;
oxidizing the waste stream in the waste treatment apparatus with an oxygen containing gas to eliminate or render inert any pharmaceutical compounds in the waste stream; and
chelating the waste stream in the waste treatment apparatus to deactivate any metal ions remaining in the waste stream.

22. The method of claim 21, wherein the oxidizing step comprises exposing the waste stream to an oxygen containing gas wherein the waste stream is at a temperature ranging from about 30° to about 90° C.

23. The method of claim 21, wherein the waste stream is conditioned in the conditioning step to provide the waste stream with a viscosity ranging from about 5 to about 200 centipoise at 20° C. and a conductivity of at least about 300 microsiemens per centimeter at 25° C.

24. The method of claim 21, wherein the iron ion generating step provides the waste stream with an iron ion concentration ranging from about 10 to about 1000 ppm by volume based on a total volume of the waste stream.
